(19) United States
(12) Patent Application Publication
(10) Pub. No.: US 2026/0226024 A1
(43) Pub. Date: Aug. 6, 2026

(54) CRYSTALLINE FORM OF ASCIMINIB HYDROCHLORIDE

(71) Applicant: Synthon B.V., Nijmegen (NL)

(72) Inventors: Bohumil DYMÁCEK, Blansko (CZ); Michal HEGEDUS, Blansko (CZ)

(21) Appl. No.: 19/128,078

(22) PCT Filed: Nov. 9, 2023
(86) PCT No.: PCT/EP2023/081342

§ 371 Date: May 7, 2025

(30) Foreign Application Priority Data
Nov. 10, 2022 (EP) 22206761.3

(51) Int. Cl. C07D 401/14

(52) U.S. Cl. CPC C07D 401/14

(57) ABSTRACT

The application relates to the crystalline Form 1S of Asciminib hydrochloride and processes for the preparation thereof. Also described are solvates of Asciminib with 2-butanol, isobutanol, tert-butanol, 2-methyl tetra hydrofuran and cyclohexanol from which the crystalline Form IS of Asciminib hydrochloride can be prepared.

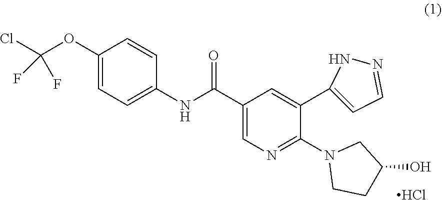 [SMILES: O=C(Nc1ccc(OC(F)(F)Cl)cc1)c1cnc(N2CC[C@@H](O)C2)c(-c2ccn[nH]2)c1 ; Cl]

The invention relates to processes for preparation of solid crystalline form 1S of Asciminib hydrochloride, and solid crystalline forms of Asciminib hydrochloride solvates.

### BACKGROUND OF THE PRESENT INVENTION

This invention relates to a solid crystalline form 1S of Asciminib hydrochloride (compound of formula (1)) and solid crystalline forms of Asciminib hydrochloride solvates, and processes for preparation thereof;

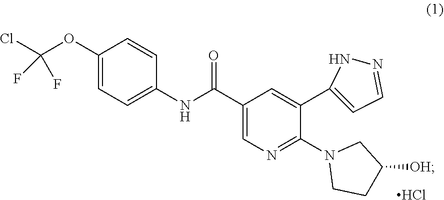 [SMILES: O=C(Nc1ccc(OC(F)(F)Cl)cc1)c1cnc(N2CC[C@@H](O)C2)c(-c2ccn[nH]2)c1 ; Cl]

Asciminib hydrochloride, N-[4-[Chloro(difluoro) methoxy]phenyl]-6-[(3R)-3-hydroxypyrrolidin-1-yl]-5-(1H-pyrazol-5-yl)pyridine-3-carboxamide hydrochloride, is an allosteric inhibitor of BCR-ABL kinase. Asciminib hydrochloride was approved and launched for the treatment of adult patients with Philadelphia chromosome-positive chronic myeloid leukemia (Ph+ CML) in chronic phase (CP), previously treated with two or more tyrosine kinase inhibitors (TKIs) and adult patients with Ph+ CML in CP with the T315I mutation. Asciminib hydrochloride was disclosed in WO 2013171639 application. Solid forms of Asciminib hydrochloride are described in WO 2021154980 or WO 2020230099 applications. It is advantageous to develop a crystalline form of Asciminib hydrochloride shows good flow properties, crystallinity, stability (incl. chemical, polymorhic or water stability), crystal shape and processability in comparison with solid forms described in prior art.

### BRIEF DESCRIPTION OF THE INVENTION

The presented invention relates to a solid crystalline form 1S of Asciminib hydrochloride (compound of formula (1)) and solid crystalline forms of Asciminib hydrochloride solvates, and processes for preparation thereof;

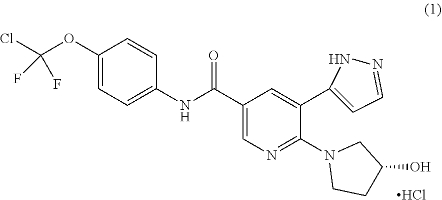 [SMILES: O=C(Nc1ccc(OC(F)(F)Cl)cc1)c1cnc(N2CC[C@@H](O)C2)c(-c2ccn[nH]2)c1 ; Cl]

The presented invention further relates to a pharmaceutical composition comprising the crystalline Form 1S of Asciminib hydrochloride.

Crystalline Form 1S of Asciminib hydrochloride of the presented invention has improved good flow properties, crystallinity, stability (including chemical, polymorphic or water stability), crystal shape and processability in comparison with solid forms described in prior art.

### BRIEF DESCRIPTION OF THE DRAWINGS

FIG. 1 depicts the X-Ray Powder Diffractogram (XRPD) of crystalline Form 1S of Asciminib hydrochloride prepared according to Example 6.

FIG. 2 depicts the X-Ray Powder Diffractogram (XRPD) of crystalline Form 1 of 2-butanol solvate of Asciminib hydrochloride prepared according to Example 1.

FIG. 3 depicts the X-Ray Powder Diffractogram (XRPD) of crystalline Form 3 of tert-butanol solvate of Asciminib hydrochloride prepared according to Example 2.

FIG. 4 depicts the X-Ray Powder Diffractogram (XRPD) of crystalline Form 4 of iso-butanol solvate of Asciminib hydrochloride prepared according to Example 3.

FIG. 5 depicts the X-Ray Powder Diffractogram (XRPD) of crystalline Form 6 of 2-methyl tertahydrofurane solvate of Asciminib hydrochloride prepared according to Example 4.

FIG. 6 depicts the X-Ray Powder Diffractogram (XRPD) of crystalline Form 2 of cyclohexanol solvate of Asciminib hydrochloride prepared according to Example 5.

FIG. 7 depicts the DSC pattern of crystalline Form 1S of Asciminib hydrochloride prepared according to Example 6.

FIG. 8 depicts the TGA pattern of crystalline Form 1S of Asciminib hydrochloride prepared according to Example 6.

FIG. 9 depicts the DSC pattern of crystalline Form 1 of 2-butanol solvate of Asciminib hydrochloride prepared according to Example 1.

FIG. 10 depicts the TGA pattern of crystalline Form 1 of 2-butanol solvate of Asciminib hydrochloride prepared according to Example 1.

FIG. 11 depicts the DSC pattern of crystalline Form 3 of tert-butanol solvate of Asciminib hydrochloride prepared according to Example 2.

FIG. 12 depicts the TGA pattern of crystalline Form 3 of tert-butanol solvate of Asciminib hydrochloride prepared according to Example 2.

FIG. 13 depicts the DSC pattern of crystalline Form 4 of iso-butanol solvate of Asciminib hydrochloride prepared according to Example 3.

FIG. 14 depicts the TGA pattern of crystalline Form 4 of iso-butanol solvate of Asciminib hydrochloride prepared according to Example 3.

FIG. 15 depicts the DSC pattern of crystalline Form 6 of 2-methyl tetrahydrofurane solvate of Asciminib hydrochloride prepared according to Example 4.

FIG. 16 depicts the TGA pattern of crystalline Form 6 of 2-methyl tetrahydrofurane solvate of Asciminib hydrochloride prepared according to Example 4.

FIG. 17 depicts the DSC pattern of crystalline Form 2 of cyclohexanol solvate of Asciminib hydrochloride prepared according to Example 5.

FIG. 18 depicts the TGA pattern of crystalline Form 2 of cyclohexanol solvate of Asciminib hydrochloride prepared according to Example 5.

FIG. 19 depicts crystal shape of solid Form 1S of Asciminib hydrochloride prepared according to Example 6.

FIG. 20 depicts crystal shape of solid Form 1S of Asciminib hydrochloride prepared according to Example 6.

FIG. 21 depicts crystal shape of solid prior art Form A of Asciminib hydrochloride

### DETAILED DESCRIPTION OF THE INVENTION

The presented invention relates to solid Form 1S of Asciminib hydrochloride, compound of formula (1), a process for preparation thereof and a formulation comprising it:

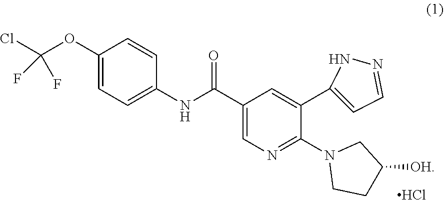 [SMILES: O=C(Nc1ccc(OC(F)(F)Cl)cc1)c1cnc(N2CC[C@@H](O)C2)c(-c2ccn[nH]2)c1 ; Cl]

The solid Form 1S can be characterized by XRPD pattern having 2θ values 5.9°, 16.7° and 19.4° degrees 2 theta (±0.2 degrees 2 theta), when measured with CuKα1 radiation (λ=1.54060 Å). The solid Form 1S can be also characterized by XRPD pattern having 2θ values 5.9°, 11.3°, 16.7°, 19.4°, 22.0° and 23.6° degrees 2 theta (±0.2 degrees 2 theta), when measured with CuKα1 radiation (λ=1.54060 Å). The solid Form 1S can be further characterized by XRPD pattern described in the following table when measured with CuKα1 radiation (λ=1.54060 Å):

| Angle | Intensity |
|---|---|
| 2Θ | % |
| 5.9 | 69.5 |
| 10.4 | 15.0 |
| 11.3 | 55.4 |
| 12.7 | 27.0 |
| 13.7 | 10.3 |
| 14.0 | 10.7 |
| 14.7 | 7.2 |
| 15.2 | 15.3 |
| 15.8 | 25.9 |
| 16.7 | 100.0 |
| 19.4 | 65.0 |
| 20.2 | 16.8 |
| 21.1 | 16.8 |
| 21.4 | 20.3 |
| 22.0 | 43.6 |
| 22.6 | 15.0 |
| 23.6 | 89.6 |
| 24.7 | 33.6 |
| 25.5 | 20.9 |
| 26.6 | 11.7 |
| 27.5 | 22.2 |
| 29.7 | 14.2 |
| 30.2 | 9.4 |
| 30.8 | 7.4 |
| 31.8 | 16.1 |
| 33.1 | 12.1 |

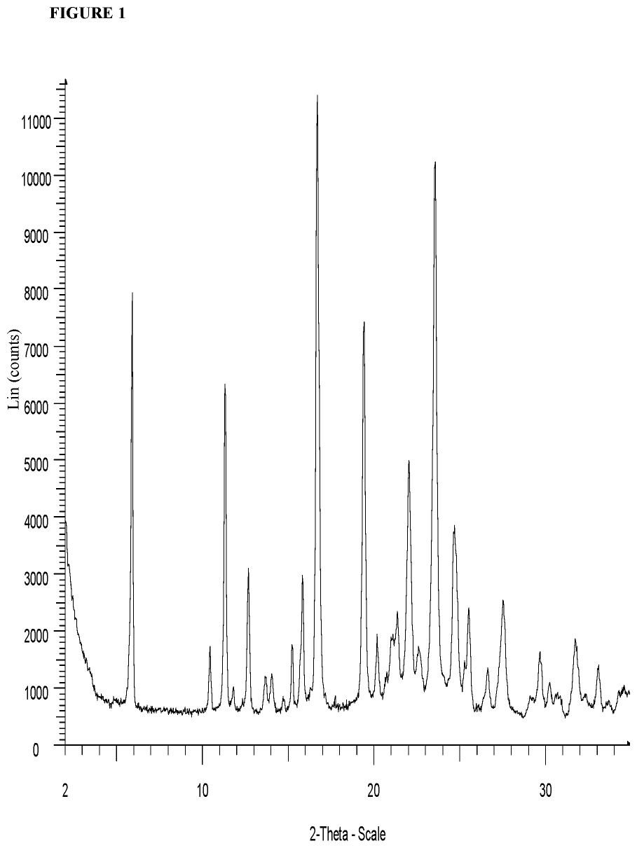

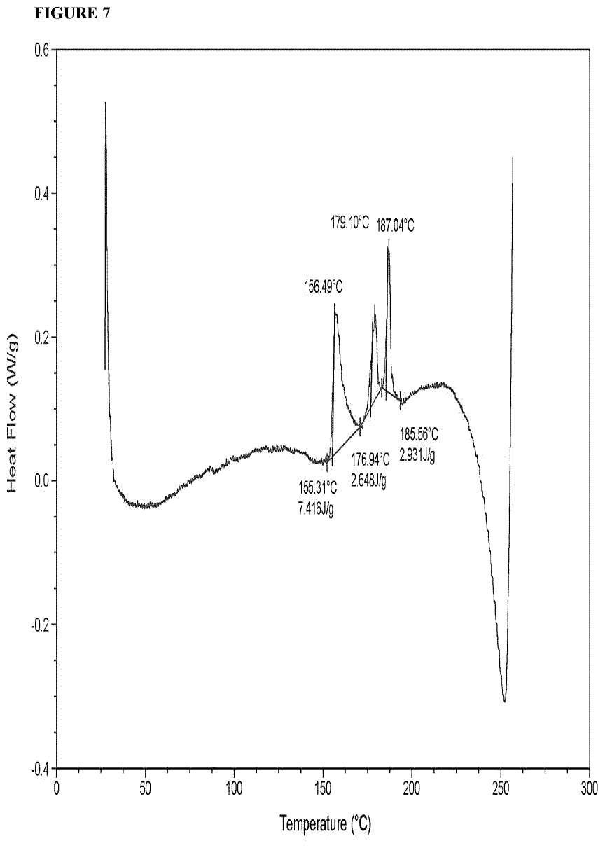

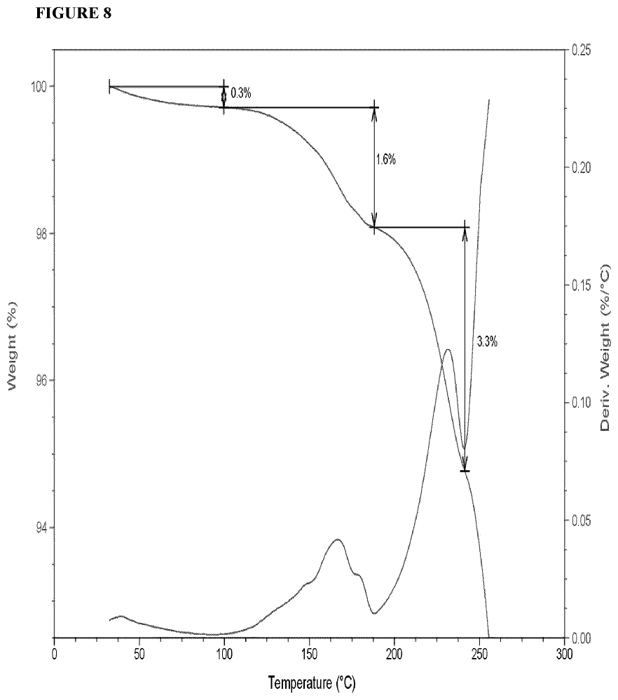

The crystalline Form 1S can be also characterized by XRPD pattern depicted in FIG. 1 or by DSC pattern depicted in FIG. 7 or TGA pattern depicted in FIG. 8.

The solid form 1S can be prepared by a process comprising drying a solvate of Asciminib hydrochloride, preferably a solvate with a solvent selected from 2-butanol or iso-butanol or tert-butanol or 2-methyl tetrahydrofurane or cyclohexanol, at a temperature between 55° C. and 65° C. for between 48 and 72 hours wherein the solvates od Asciminib hydrochloride can be defined as crystallizing in the triclinic crystal system, with two of their unit cell parameters between 8-9.5 angstroms and the third unit cell parameter between 17.5-19 angstroms. The profile fitting and indexation were carried out in FOXgrid software. The indexation of the unit cell was performed from first 15 peaks using dichotomy algorithm. All possible crystal systems were explored with restrictions on unit cell volumes. The solution with highest figure of merit was selected and LeBail refinement was performed in JANA 2020 software. For the refinement, P-V profile function was utilized. Except for unit cell parameters, sample offset, profile parameters and background coefficients were refined. The solutions were then evaluated based on GoF and R factors, which represent reliability factors for the refinement.

The solvate of Asciminib hydrochloride, preferably a solvate with a solvent selected from 2-butanol or iso-butanol or tert-butanol or 2-methyl tetrahydrofurane or cyclohexanol, wherein the solvates can be defined as crystallizing in the triclinic crystal system, with two of their unit cell parameters between 8-9.5 angstroms and the third unit cell parameter between 17.5-19 angstroms, can be prepared by a process comprising:
1. Suspending of Asciminib hydrochloride, preferably amorphous form thereof, in the solvent;
2. Stirring the mixture at a temperature between 20° C. and 30° C. for between 20 and 120 minutes;
3. Isolating the solvate of Asciminib hydrochloride.

All prepared Asciminib hydrochloride solvates, preferably solvates with a solvent selected from with 2-butanol or iso-butanol or tert-butanol or 2-methyl tetrahydrofurane or cyclohexanol are isostructural compounds. Isostructural compounds, in general, are the compounds with different chemical composition which however have similar crystal structures and packing patterns in their solid form. Subsequently, compounds having similar/identical arrangement of building blocks exhibit similar diffraction patterns. Since the position of diffractions peaks is determined by unit cell parameters, isostructural compounds exhibiting similar diffraction patterns, will also have close to identical unit cell parameters. Asciminib hydrochloride solvates prepared by presented process can be defined as crystallizing in the triclinic crystal system, with two of their unit cell parameters between 8-9.5 angstroms and the third unit cell parameter between 17.5-19 angstroms.

The concentration of Asciminib hydrochloride, preferably amorphous form thereof, in the solvent can be between 0.07 g/ml and 0.5 g/ml. Asciminib hydrochloride, preferably amorphous form thereof, is suspended in the solvent and the mixture is stirred at a temperature between 20° C. and 30° C. for between 20 and 120 minutes. The obtained solid can be isolated by any suitable technique, for example using filtration to provide the solvate of Asciminib hydrochloride.

The solid form of solvate of Asciminib hydrochloride with 2-butanol, Form 1, can be characterized by XRPD pattern having 2θ values 4.9°, 10.3° and 16.6° degrees 2 theta (+0.2 degrees 2 theta), when measured with CuKα1 radiation (λ2=1.54060 Å). The solid form can be also characterized by XRPD pattern having 2θ values 4.9°, 10.3°, 15.0°, 16.6° and 22.1° degrees 2 theta (+0.2 degrees 2 theta), when measured with CuKα1 radiation (λ2=1.54060 Å). The solid Form 1 can be further characterized by XRPD pattern described in the following table when measured with CuKα1 radiation (%=1.54060 Å):

| Angle | Intensity |
|---|---|
| 2Θ | % |
| 4.9 | 100.0 |
| 9.9 | 16.2 |
| 10.3 | 51.2 |
| 10.9 | 21.6 |
| 12.0 | 4.6 |
| 13.3 | 21.9 |
| 15.0 | 33.3 |
| 15.8 | 20.1 |
| 16.6 | 91.8 |
| 16.9 | 15.3 |
| 18.0 | 7.1 |
| 19.1 | 5.3 |
| 20.6 | 12.5 |
| 21.1 | 26.4 |
| 21.5 | 48.0 |
| 21.7 | 49.6 |
| 22.1 | 86.6 |
| 22.9 | 7.9 |
| 23.6 | 23.4 |
| 24.0 | 29.9 |
| 24.7 | 11.3 |
| 25.3 | 7.1 |
| 25.6 | 11.1 |
| 26.7 | 21.1 |
| 27.1 | 7.4 |
| 28.0 | 11.0 |
| 28.3 | 7.5 |
| 29.4 | 4.0 |
| 30.2 | 7.5 |
| 30.7 | 11.0 |
| 31.2 | 6.9 |
| 31.8 | 4.4 |
| 32.4 | 7.8 |
| 33.0 | 8.2 |
| 34.1 | 5.3 |
| 34.6 | 7.6 |

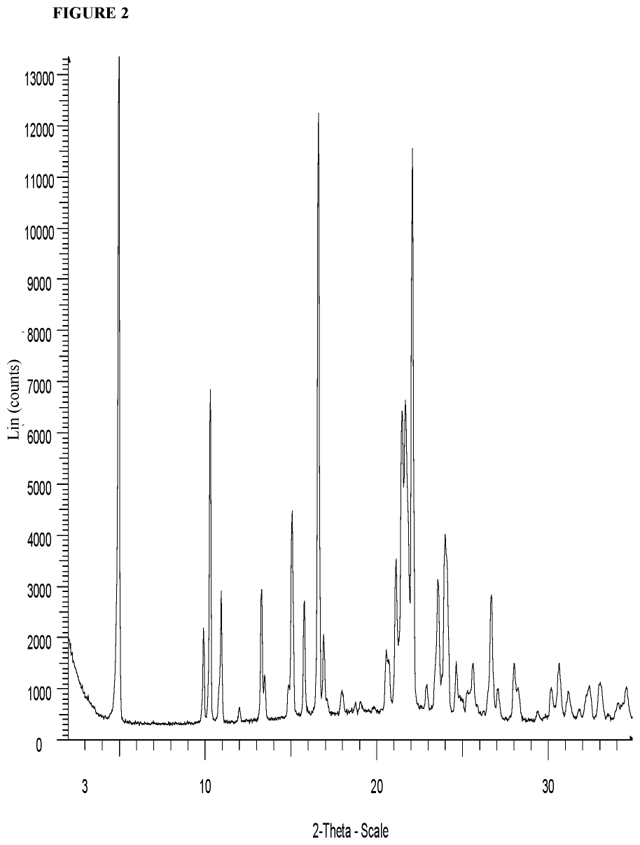

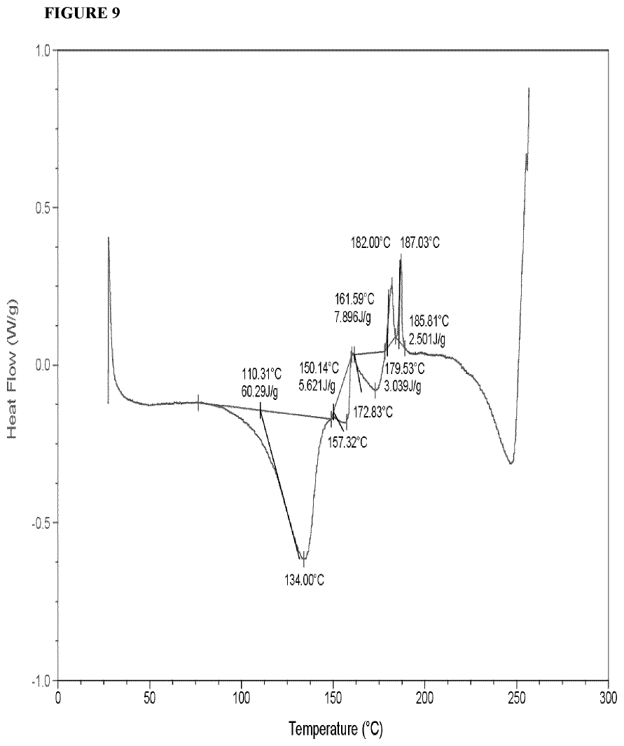

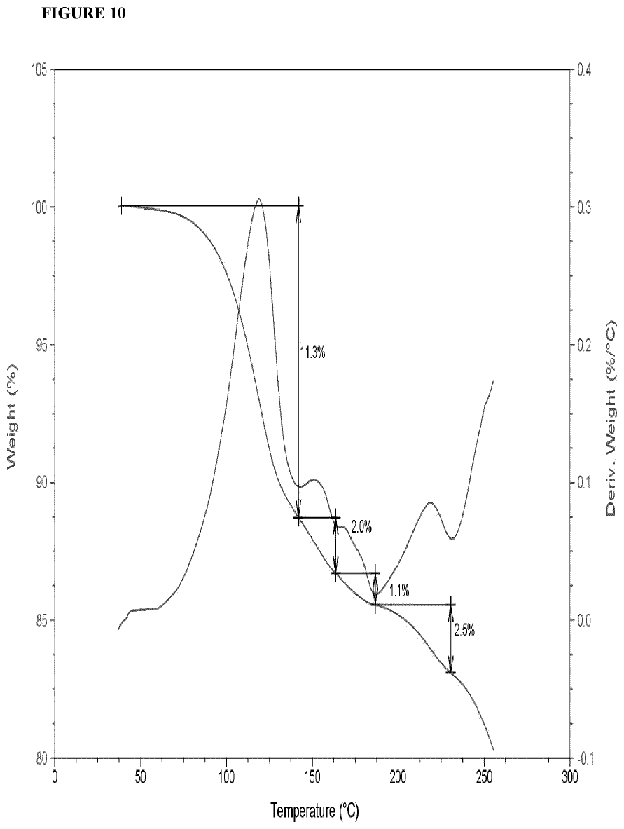

The crystalline Form 1 can be also characterized by XRPD pattern depicted in FIG. 2 or by DSC pattern depicted in FIG. 9 or TGA pattern depicted in FIG. 10.

The solid form of solvate of Asciminib hydrochloride with tert-butanol, Form 3, can be characterized by XRPD pattern having 2θ values 5.0°, 10.5° and 21.8° degrees 2 theta (+0.2 degrees 2 theta), when measured with CuKα1 radiation (λ=1.54060 Å). The solid form can be also characterized by XRPD pattern having 2θ values 5.0°, 10.5°, 16.8°, 21.4° and 21.8° degrees 2 theta (±0.2 degrees 2 theta), when measured with CuKα1 radiation (%=1.54060 Å). The solid Form 3 can be further characterized by XRPD pattern described in the following table when measured with CuKα1 radiation (λ=1.54060 Å):

| Angle | Intensity |
|---|---|
| 2Θ | % |
| 5.0 | 51.0 |
| 9.9 | 14.3 |
| 10.5 | 40.6 |
| 10.8 | 19.8 |
| 13.1 | 15.0 |
| 13.3 | 11.2 |
| 15.0 | 18.3 |
| 15.2 | 21.2 |
| 15.9 | 13.6 |
| 16.6 | 22.7 |
| 16.8 | 68.5 |
| 17.6 | 9.7 |
| 18.3 | 9.5 |
| 19.1 | 11.6 |
| 20.8 | 13.4 |
| 21.4 | 51.2 |
| 21.8 | 100.0 |
| 22.2 | 16.4 |
| 23.7 | 28.5 |
| 24.1 | 27.7 |
| 25.0 | 11.4 |
| 25.3 | 10.3 |
| 25.9 | 8.0 |
| 26.4 | 14.9 |
| 27.7 | 15.0 |
| 28.0 | 6.9 |
| 28.5 | 5.4 |
| 28.8 | 5.9 |
| 29.7 | 4.3 |
| 30.9 | 6.0 |
| 31.6 | 9.9 |
| 32.1 | 8.8 |
| 32.5 | 9.2 |
| 33.6 | 6.9 |
| 34.1 | 4.9 |
| 34.7 | 6.2 |

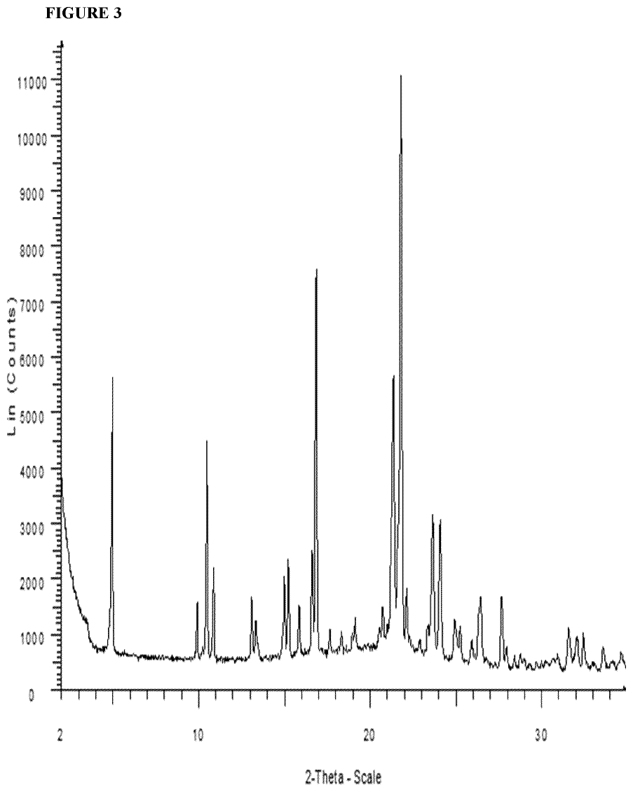

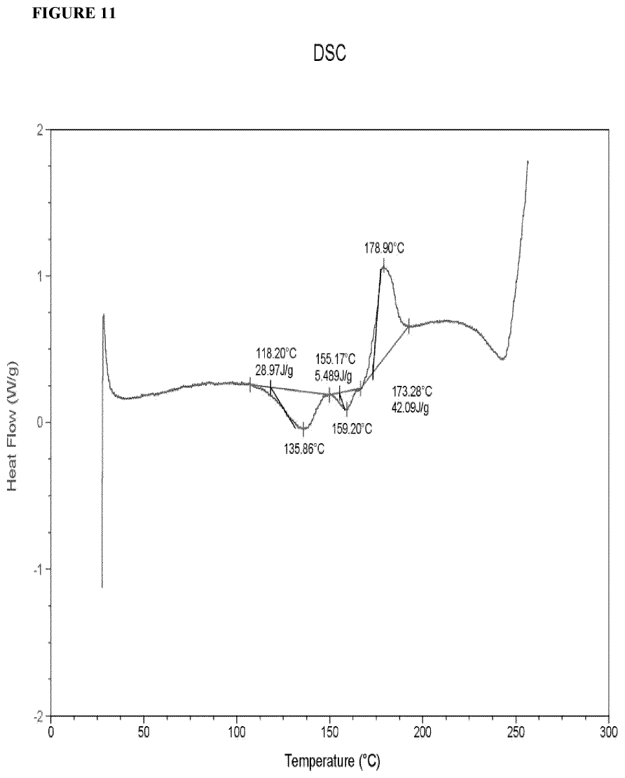

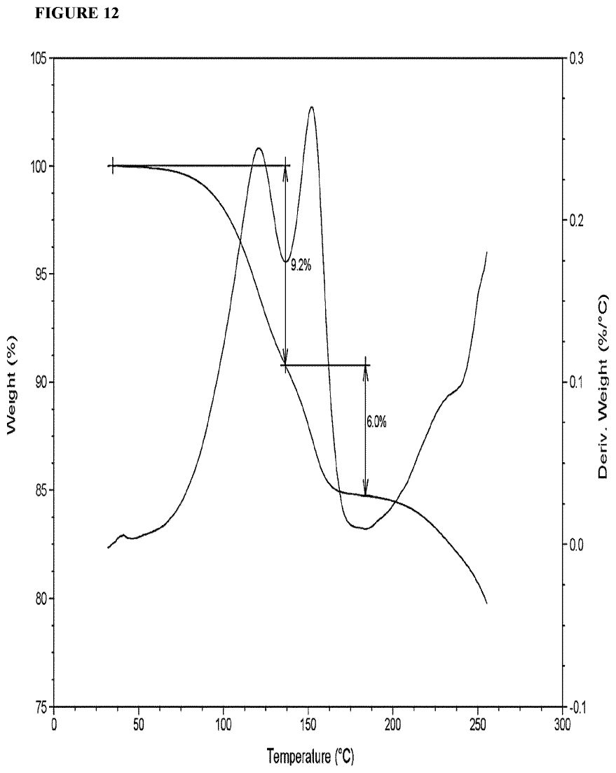

The crystalline Form 3 can be also characterized by XRPD pattern depicted in FIG. 3 or by DSC pattern depicted in FIG. 11 or TGA pattern depicted in FIG. 12.

The solid form of solvate of Asciminib hydrochloride with iso-butanol, Form 4, can be characterized by XRPD pattern having 2θ values 10.2°, 16.6° and 22.1° degrees 2 theta (+0.2 degrees 2 theta), when measured with CuKα1 radiation (λ=1.54060 Å). The solid form can be also characterized by XRPD pattern having 2θ values 4.9°, 10.2°, 16.6°, 21.2° and 22.1° degrees 2 theta (+0.2 degrees 2 theta), when measured with CuKα1 radiation (λ=1.54060 Å). The solid Form 4 can be further characterized by XRPD pattern described in the following table when measured with CuKα1 radiation (λ=1.54060 Å):

| Angle | Intensity |
|---|---|
| 2Θ | % |
| 4.9 | 23.7 |
| 9.9 | 6.9 |
| 10.2 | 52.2 |
| 11.0 | 13.4 |
| 11.8 | 5.1 |
| 13.1 | 15.2 |
| 14.7 | 17.5 |
| 15.0 | 12.0 |
| 15.8 | 19.3 |
| 16.6 | 79.0 |
| 17.0 | 8.1 |
| 18.2 | 5.6 |
| 18.5 | 5.4 |
| 20.5 | 21.0 |
| 20.9 | 21.7 |
| 21.2 | 42.9 |
| 21.6 | 23.8 |
| 22.1 | 100.0 |
| 22.5 | 7.6 |
| 23.3 | 6.8 |
| 23.5 | 9.8 |
| 23.9 | 8.2 |
| 24.2 | 14.9 |
| 24.4 | 16.6 |
| 24.8 | 8.5 |
| 25.1 | 5.5 |
| 25.4 | 6.8 |
| 25.8 | 5.6 |
| 26.5 | 13.1 |
| 26.9 | 7.8 |
| 28.0 | 10.2 |
| 30.4 | 7.4 |
| 30.9 | 4.7 |
| 31.9 | 6.1 |
| 32.6 | 5.4 |
| 33.4 | 6.8 |
| 33.7 | 4.2 |
| 34.5 | 4.8 |

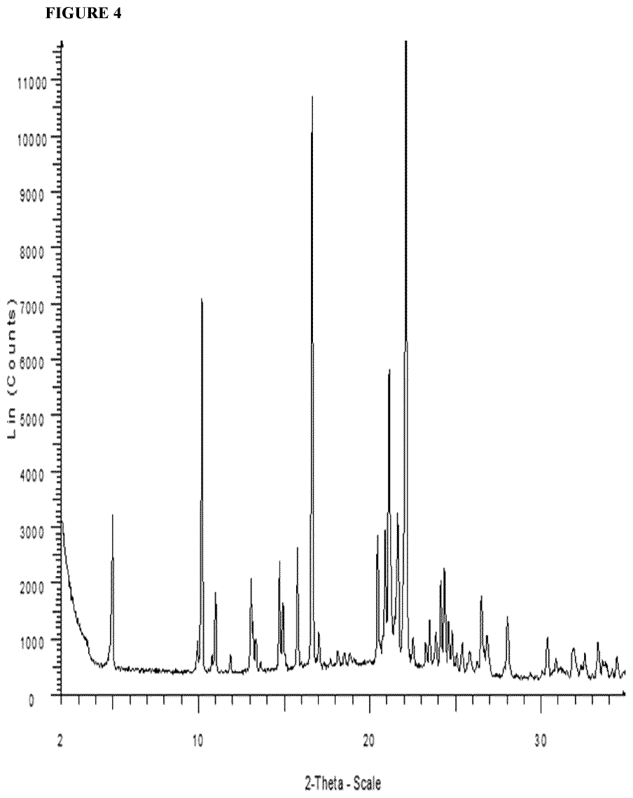

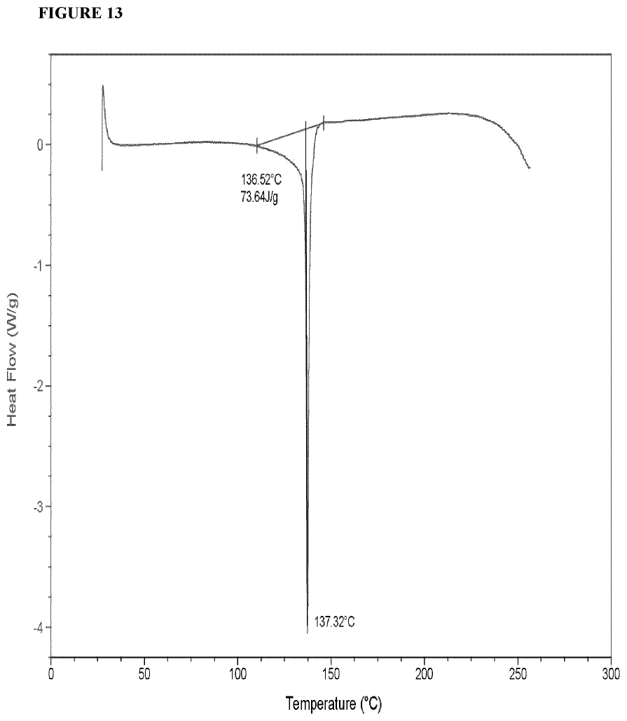

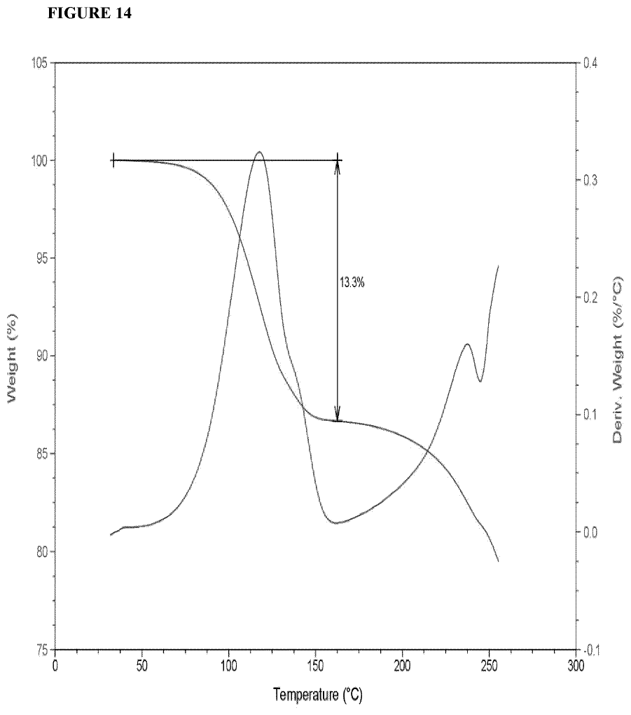

The crystalline Form 4 can be also characterized by XRPD pattern depicted in FIG. 4 or by DSC pattern depicted in FIG. 13 or TGA pattern depicted in FIG. 14.

The solid form of solvate of Asciminib hydrochloride with 2-methyl tetrahydrofurane, Form 6, can be characterized by XRPD pattern having 2θ values 4.9°, 16.9° and 21.9° degrees 2 theta (+0.2 degrees 2 theta), when measured with CuKα1 radiation (λ=1.54060 Å). The solid form can be also characterized by XRPD pattern having 2θ values 4.9°, 10.4°, 16.9°, 20.9° and 21.9° degrees 2 theta (±0.2 degrees 2 theta), when measured with CuKα1 radiation (λ2=1.54060 Å). The solid Form 6 can be further characterized by XRPD pattern described in the following table when measured with CuKα1 radiation (λ=1.54060 Å):

| Angle | Intensity |
|---|---|
| 2Θ | % |
| 4.9 | 87.8 |
| 9.8 | 25.2 |
| 10.4 | 55.5 |
| 11.0 | 18.2 |
| 12.4 | 9.2 |
| 12.7 | 25.7 |
| 13.4 | 10.4 |
| 15.1 | 21.3 |
| 15.3 | 30.5 |
| 16.1 | 32.2 |
| 16.9 | 92.4 |
| 18.4 | 13.2 |
| 19.2 | 19.3 |
| 20.4 | 16.0 |
| 20.9 | 64.7 |
| 21.7 | 63.0 |
| 21.9 | 100.0 |
| 22.2 | 24.1 |
| 22.9 | 14.2 |
| 23.3 | 14.6 |
| 23.6 | 30.3 |
| 24.1 | 33.8 |
| 24.7 | 11.1 |
| 25.5 | 12.1 |
| 26.3 | 15.4 |
| 27.4 | 14.4 |
| 28.0 | 4.7 |
| 28.7 | 7.7 |
| 29.3 | 6.1 |
| 29.7 | 2.8 |
| 30.3 | 4.6 |
| 30.5 | 4.3 |
| 30.9 | 4.1 |
| 31.8 | 12.3 |
| 32.3 | 6.3 |
| 32.9 | 4.9 |
| 34.0 | 3.5 |
| 34.7 | 3.8 |

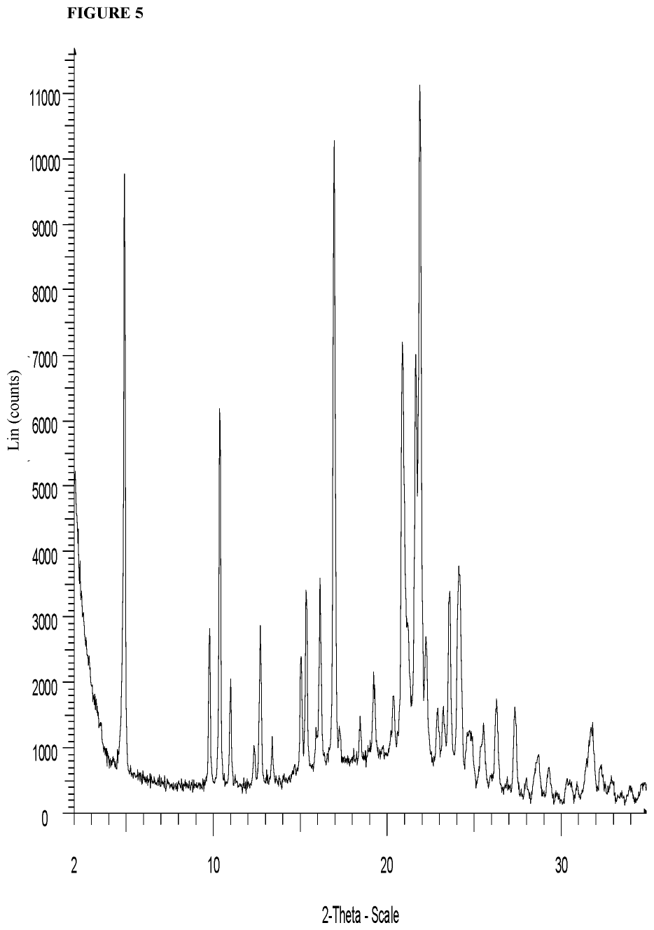

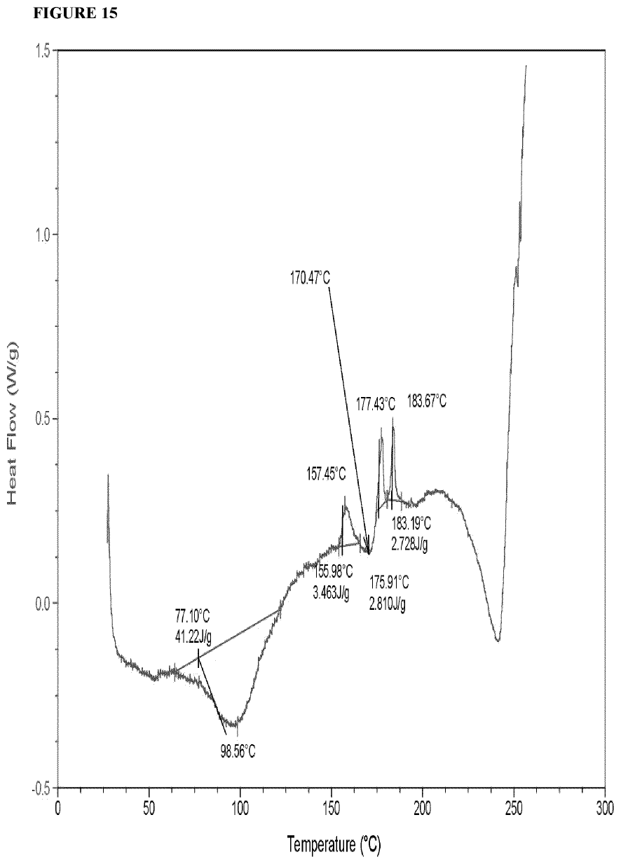

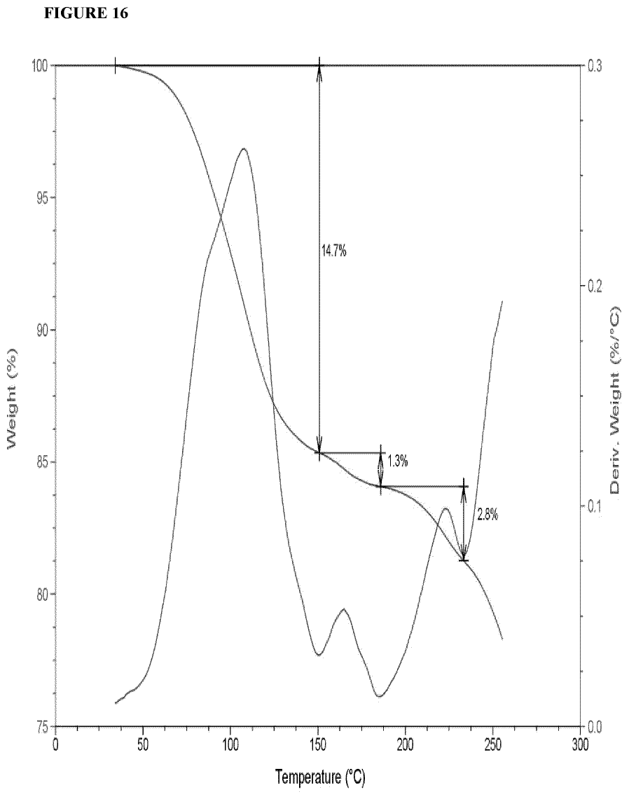

The crystalline Form 6 can be also characterized by XRPD pattern depicted in FIG. 5 or by DSC pattern depicted in FIG. 15 or TGA pattern depicted in FIG. 16.

The solid form of solvate of Asciminib hydrochloride with cyclohexanol, Form 2, can be characterized by XRPD pattern having 2θ values 10.1°, 16.5° and 21.5° degrees 2 theta (+0.2 degrees 2 theta), when measured with CuKα1 radiation (λ2=1.54060 Å). The solid form can be also characterized by XRPD pattern having 2θ values 4.9°, 10.1°, 15.9°, 16.5° and 21.5° degrees 2 theta (+0.2 degrees 2 theta), when measured with CuKα1 radiation (λ=1.54060 Å). The solid Form 6 can be further characterized by XRPD pattern described in the following table when measured with CuKα1 radiation (λ=1.54060 Å):

| Angle | Intensity |
|---|---|
| 2Θ | % |
| 4.9 | 15.0 |
| 9.8 | 6.4 |
| 10.1 | 34.9 |
| 10.8 | 9.0 |
| 11.7 | 9.9 |
| 12.7 | 6.3 |
| 13.5 | 9.3 |
| 14.4 | 15.0 |
| 14.8 | 5.7 |
| 14.9 | 11.6 |
| 15.9 | 22.5 |
| 16.5 | 71.4 |
| 17.2 | 9.7 |
| 18.8 | 6.3 |
| 20.7 | 42.6 |
| 21.1 | 8.6 |
| 21.5 | 100.0 |
| 21.9 | 9.6 |
| 23.0 | 8.7 |
| 23.2 | 6.1 |
| 23.5 | 19.4 |
| 23.8 | 14.8 |
| 24.3 | 7.8 |
| 24.7 | 8.6 |
| 25.3 | 5.8 |
| 25.9 | 12.0 |
| 26.4 | 5.1 |
| 27.1 | 4.8 |
| 27.4 | 8.5 |
| 29.8 | 6.6 |
| 30.8 | 4.5 |
| 31.3 | 5.3 |
| 31.8 | 6.2 |
| 32.3 | 4.1 |
| 32.7 | 6.5 |
| 33.3 | 3.2 |
| 33.7 | 3.5 |
| 34.1 | 4.0 |
| 34.7 | 4.8 |

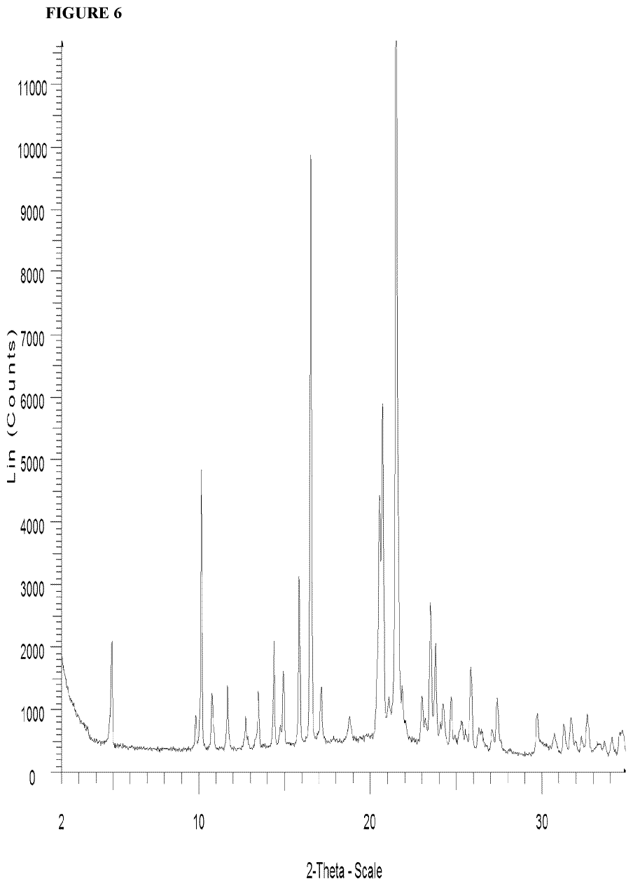

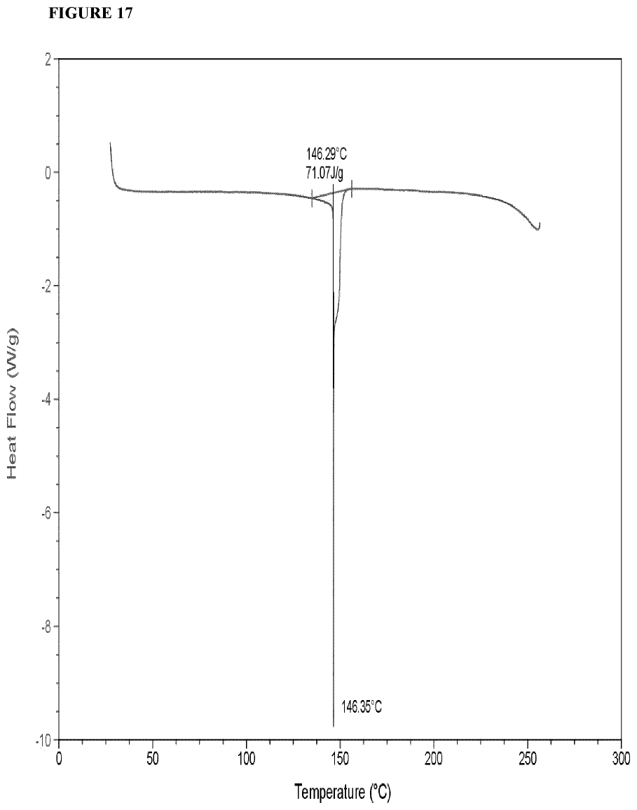

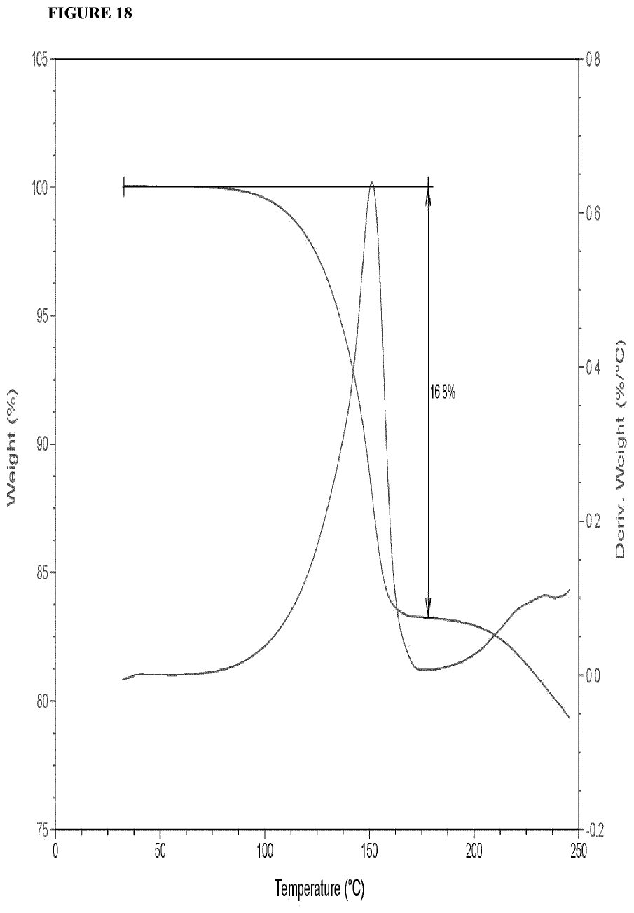

The crystalline Form 2 can be also characterized by XRPD pattern depicted in FIG. 6 or by DSC pattern depicted in FIG. 17 or TGA pattern depicted in FIG. 18.

Amorphous Asciminib hydrochloride can be prepared by a process comprising:
1. Dissolving Asciminib in an C₁ to C₄ alcohol, preferably in methanol;
2. Adding concentrated hydrochloride acid and water, wherein the volume ratio between the alcohol and water is between 8:1 and 12:1, preferably it is between 9:1 and 10:1;
3. Concentrating the mixture.
C₁ to C₄ alcohol can be selected form methanol or ethanol or propanol or isopropanol or butanol or 2-butanol or iso-butanol or tert-butanol, preferably it is methanol. The concentration of Asciminib in C₁ to C₄ alcohol can be between 0.07 g/ml and 0.15 g/ml, preferably it is between 0.09 g/ml and 0.11 g/ml. The mixture is heated to a temperature between 40° C. and 60° C. to obtain a solution. To the solution concentrated (35% vol/vol) hydrochloric acid is added. The molar ratio between hydrochloric acid and Asciminib can be between 3:1 and 3.5:1. To the mixture water is added, wherein the volume ratio between the alcohol and water is between 8:1 and 12:1, preferably it is between 9:1 and 10:1. Obtained mixture is concentrated, preferably at vacuum to obtain amorphous Asciminib hydrochloride.

The crystalline forms according to presented invention, preferably Form 1S, of Asciminib hydrochloride or a solvate thereof, can be processed into a suitable pharmaceutical formulation. In the pharmaceutical formulation the solid forms can be mixed with pharmaceutically acceptable adjuvants, diluents or carriers. The amount of crystalline forms prepared according to presented invention, preferably Form 1S, in the formulation depends on the condition and a patient to be treated. The pharmaceutical formulation can be if form of a solid oral formulation, for example a capsule, a pill, a powder or a granule. In the formulation the crystalline forms prepared according to presented invention, preferably Form 1S, can be mixed with one or more additives such as fillers or extenders or binders or wetting agents or disintegrating agents or absorbents or lubricants or buffering agents. The formulation in a form of a tablet or a dragee or a capsule or a pill or a granule can be coated with a coating or shell such as enteric or other coating. The oral formulation can be in a form of an oral emulsion or a solution or a suspension or a syrup. The formulation can contain suitable additives such as diluent(s) or wetting agent(s) or emulsifying agent(s) or suspending agent(s) or sweetening agent(s) or flavouring agent(s). The examples of suitable additive(s) are known to those skilled in the art.

The suitable pharmaceutical formulation can be in a parenteral form such as an injection or an infusion or an injectable depot or in a liposomal form comprising pharmaceutically acceptable aqueous or non-aqueous solution(s) or dispersion(s) or emulsions. The pharmaceutical formulation can be also in a form of a powder for reconstitution into an injection or infusion. The formulation can further comprise additives such as preservative(s) or wetting agent(s) or emulsifying agent(s) or dispersing agent(s) or antibacterial or anti-fungal agents. The examples of suitable additive(s) are known to those skilled in the art.

The suitable pharmaceutical formulation can be in a form suitable for rectal or vaginal administration further comprising suitable additive(s). The examples of suitable additive(s) are known to those skilled in the art.

The crystalline forms according to presented invention, preferably Form 1S, of Asciminib hydrochloride or a solvate thereof, a pharmaceutical formulation comprising the forms can be used for the treatment of conditions treatable with Asciminib or a salt thereof.

### EXAMPLES

Asciminib hydrochloride was prepared according to a process disclosed in application WO2013/171639, Asciminib hydrochloride amorphous form thereof can be prepared according to processes described in Example 7.
DCS/TGA patterns were obtained using the following conditions: 10° C./min->260° C. Pictures of crystals were obtained by SEM Tescan Vega.
XRPD spectrum was obtained using the following measurement conditions: Panalytical Empyrean diffractometer with Θ/2Θ geometry (transmission mode), equipped with a PixCell 3D detector;

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406 Å (Kα1) | |
| Divergence slit: | ½° | |
| Antiscatter slit: | ½° | |
| Soller slit: | 0.02 | rad |
| Detector slit: | 7.5 | mm |
| Rotation speed: | 30 | rpm |

### Example 1: Preparation of Crystalline Form 1 of 2-Butanol Solvate of Asciminib Hydrochloride

500 mg of amorphous Asciminib hydrochloride was suspended at 25° C. in 5 ml of 2-butanol. The mixture was stirred for 30 minutes and filtered to provide solid Form 1 of 2-butanol solvate of Asciminib hydrochloride in almost 100% yield.

The prepared crystalline Form 1 can be characterized by XRPD pattern depicted in FIG. 2 or by DSC pattern depicted in FIG. 9 or TGA pattern depicted in FIG. 10.

### Example 2: Preparation of Crystalline Form 3 of Tert-Butanol Solvate of Asciminib Hydrochloride

500 mg of amorphous Asciminib hydrochloride was suspended at 25° C. in 5 ml of tert-butanol. The mixture was stirred for 30 minutes and filtered to provide solid Form 3 of tert-butanol solvate of Asciminib hydrochloride in almost 100% yield.

The prepared crystalline Form 3 can be characterized by XRPD pattern depicted in FIG. 3 or by DSC pattern depicted in FIG. 11 or TGA pattern depicted in FIG. 12.

### Example 3: Preparation of Crystalline Form 4 of Iso-Butanol Solvate of Asciminib Hydrochloride

500 mg of amorphous Asciminib hydrochloride was suspended at 25° C. in 5 ml of iso-butanol. The mixture was stirred for 30 minutes and filtered to provide solid Form 4 of iso-butanol solvate of Asciminib hydrochloride in almost 100% yield.

The prepared crystalline Form 4 can be characterized by XRPD pattern depicted in FIG. 4 or by DSC pattern depicted in FIG. 13 or TGA pattern depicted in FIG. 14.

### Example 4: Preparation of Crystalline Form 6 of 2-Methyl Tetrahydrofurane Solvate of Asciminib Hydrochloride

500 mg of amorphous Asciminib hydrochloride was suspended at 25° C. in 5 ml of 2-methyl tetrahydrofurane. The mixture was stirred for 30 minutes and filtered to provide solid Form 6 of 2-methyl tertahydrofurane solvate of Asciminib hydrochloride in almost 100% yield.

The prepared crystalline Form 6 can be characterized by XRPD pattern depicted in FIG. 5 or by DSC pattern depicted in FIG. 15 or TGA pattern depicted in FIG. 16.

### Example 5: Preparation of Crystalline Form 2 of Cyclohexanol Solvate of Asciminib Hydrochloride

500 mg of amorphous Asciminib hydrochloride was suspended at 25° C. in 5 ml of cyclohexanol. The mixture was stirred for 30 minutes and filtered to provide solid Form 2 of cyclohexanol solvate of Asciminib hydrochloride in almost 100% yield.

The prepared crystalline Form 2 can be also characterized by XRPD pattern depicted in FIG. 6 or by DSC pattern depicted in FIG. 17 or TGA pattern depicted in FIG. 18.

### Example 6: Preparation of Crystalline Form 1S of Asciminib Hydrochloride

The solid forms prepared according to Examples 1 to 6 were dried on air at 25° C. for 15 hours and then dried at 60° C. for 90 hours to provide Form 1S of Asciminib hydrochloride.

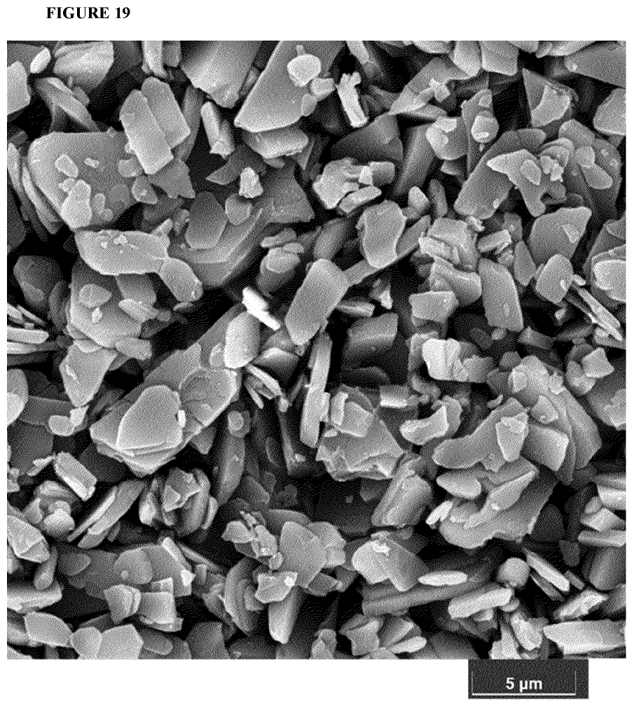

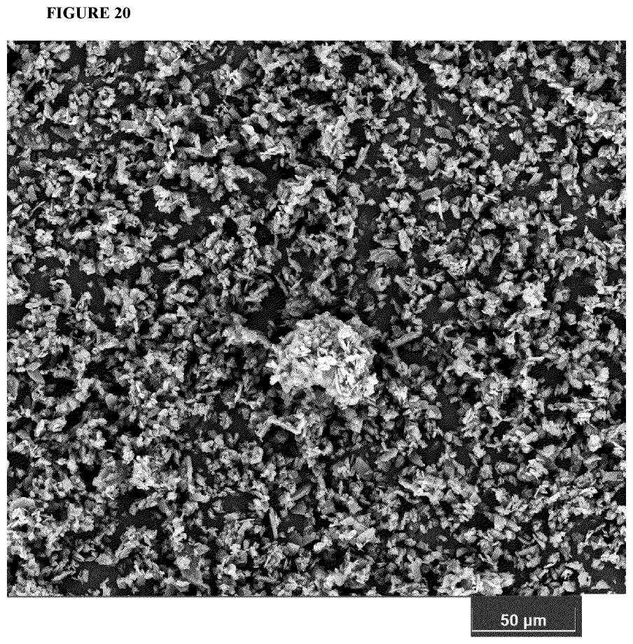

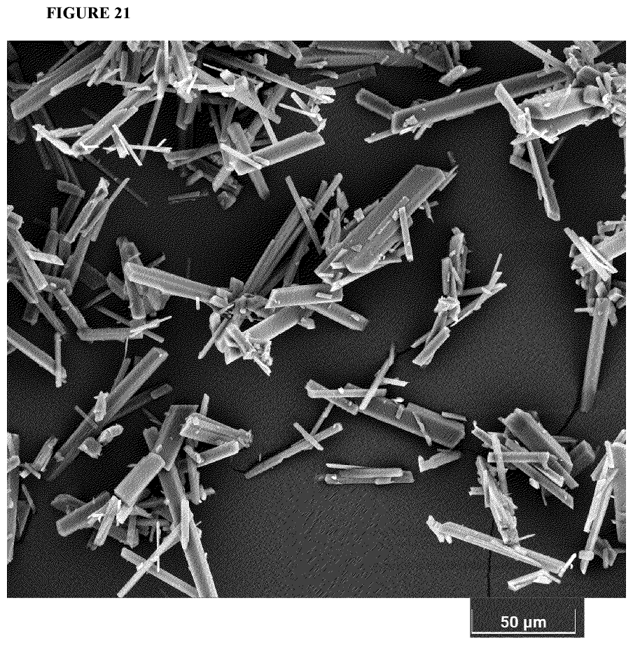

The prepared crystalline Form 1S can be also characterized by XRPD pattern depicted in FIG. 1 or by DSC pattern depicted in FIG. 7 or TGA pattern depicted in FIG. 8. The crystal shape of prepared Form 1S is depicted in FIGS. 19 and 20. The crystal shape of prior art Form A is depicted in FIG. 21. If can be concluded that crystal shape (uniform crystals) of Form 1S in comparison with Form A (needle shaped crystals) will provide better processability of Solid Form 1S in view of both processability of solid Form 1S and processability of solid Form 1S into a final product comprising it.

### Example 7: Preparation of Amorphous Form of Asciminib Hydrochloride

5 g of Asciminib was suspended in 50 ml of methanol at 20-25° C. The mixture was stirred and heated to 50° C. to obtain a solution. To the solution 1.331 g of concentrated (35%) hydrochloric acid and 5 ml of water were added. The mixture was concentrated at vacuum (2-20 kPa/60° C.) to dryness to obtain amorphous form of Asciminib hydrochloride.

### Example 8: Unit Cell Parameters of Asciminib Hydrochloride Solvates

In the table unit cell parameters of Asciminib hydrochloride solvates prepared according to Examples 1 to 5 are summarized (Rwp factors are given in %):

| | | | | | | | |
|---|---|---|---|---|---|---|---|
| a (Å) | b (Å) | c (Å) | α (°) | β (°) | γ (°) | GoF | Rwp |

2-butanol solvate, Form 1

| | | | | | | | | |
|---|---|---|---|---|---|---|---|---|
| Form | 18.285482 | 8.873341 | 8.816487 | 100.8139 | 82.0339 | 98.9521 | 1.04 | 4.18 |

Cyclohexanol solvate, Form 2

| | | | | | | | |
|---|---|---|---|---|---|---|---|
| 18.221785 | 8.894143 | 8.784646 | 100.6963 | 82.2897 | 98.6805 | 1.49 | 5.52 |

Tert-butanol solvate, Form 3

| | | | | | | | |
|---|---|---|---|---|---|---|---|
| 18.344488 | 8.880774 | 8.753618 | 101.3301 | 78.4461 | 98.2689 | 1.16 | 3.82 |

Iso-butanol solvate, Form 4

| | | | | | | | |
|---|---|---|---|---|---|---|---|
| 18.088591 | 8.925744 | 8.831858 | 101.4893 | 83.2881 | 98.0919 | 1.67 | 6.08 |

2-methyl tetrahydrofurane solvate, Form 6

| | | | | | | | |
|---|---|---|---|---|---|---|---|
| 18.665752 | 8.723692 | 8.875395 | 78.6019 | 103.3461 | 98.671 | 2.06 | 7.52 |

Asciminib hydrochloride, Form 1S

| | | | | | | | |
|---|---|---|---|---|---|---|---|
| 15.142776 | 8.597144 | 8.572371 | 98.9549 | 84.9426 | 98.205 | 1.43 | 5.20 |

1. A solid form 1S of Asciminib hydrochloride characterized by XRPD pattern having 2θ values 5.9°, 16.7° and 19.4° degrees 2 theta (+0.2 degrees 2 theta), when measured with CuKα1 radiation (λ=1.54060 Å).

2. The solid form according to claim 1 characterized by XRPD pattern having 2θ values 5.9°, 11.3°, 16.7°, 19.4°, 22.0° and 23.6° degrees 2 theta (±0.2 degrees 2 theta), when measured with CuKα1 radiation (λ=1.54060 Å).

3. A process for preparation of solid form according to claim 1, the process comprising drying a solid form of solvate of Asciminib hydrochloride at a temperature between 55° C. and 65° C. for between 48 and 72 hours wherein the solvates can be defined as crystallizing in the triclinic crystal system, with two of their unit cell parameters between 8-9.5 angstroms and the third unit cell parameter between 17.5-19 angstroms.

4. The process according to claim 3 wherein the solvate is selected from 2-butanol or iso-butanol ort tert-butanol or 2-methyl tetrahydrofurane or cyclohexanol.

5. A process for preparation of solid form according to claim 1, the process comprising drying a solid form of solvate of Asciminib hydrochloride at a temperature between 55° C. and 65° C. for between 48 and 72 hours wherein the solvate is selected from 2-butanol or iso-butanol or tert-butanol or 2-methyl tetrahydrofurane or cyclohexanol.

6. The process according to claim 5 wherein the solid form of 2-butanol solvate is characterized by XRPD pattern having 2θ values 4.9°, 10.3° and 16.6° degrees 2 theta (±0.2 degrees 2 theta), when measured with CuKα1 radiation (λ=1.54060 Å).

7. The process according to claim 5 wherein the solid form of iso-butanol solvate is characterized by XRPD pattern having 2θ values 10.2°, 16.6° and 22.1° degrees 2 theta (±0.2 degrees 2 theta), when measured with CuKα1 radiation (λ=1.54060 Å).

8. The process according to claim 5 wherein the solid form of tert-butanol solvate is characterized by XRPD pattern having 2θ values 5.0°, 10.5° and 21.8° degrees 2 theta (±0.2 degrees 2 theta), when measured with CuKα1 radiation (λ=1.54060 Å).

9. The process according to claim 5 wherein the solid form of 2-methyl tetrahydrofurane solvate is characterized by XRPD pattern having 2θ values 4.9°, 16.9° and 21.9° degrees 2 theta (±0.2 degrees 2 theta), when measured with CuKα1 radiation (λ=1.54060 Å).

10. The process according to claim 5 wherein the solid form of cyclohexanol solvate is characterized by XRPD pattern having 2θ values 10.1°, 16.5° and 21.5° degrees 2 theta (±0.2 degrees 2 theta), when measured with CuKα1 radiation (λ=1.54060 Å).
